(19) United States
(12) Patent Application Publication
(10) Pub. No.: US 2012/0165825 A1
(43) Pub. Date: Jun. 28, 2012

(54) CIRC CLIP

(76) Inventor: Ellyn Theophilopoulos, Tarpon Springs, FL (US)

(21) Appl. No.: 12/979,173

(22) Filed: Dec. 27, 2010

(51) Int. Cl. A61B 17/326

(52) U.S. Cl. 606/118

(57) ABSTRACT

A circumcision clip is provided with a first clip and a second clip coupled together by way of a retracting mechanism. The first clip and the second clip have a flat surface contiguous with an underside of the retracting mechanism to lay flat against a bell once the clips are securely fastened to the foreskin edges. This allows the circumcision clip to lay snugly against the bell once the circumcision clip is fastened to the foreskin edges. Other features are disclosed in various embodiments.

### FIELD

The present invention relates to surgical devices and, more particularly, to a circumcision instrument.

### BACKGROUND

Circumcisions are a common procedure done during the newborn period; approximately 65% of all male newborn infants undergo a circumcision. Potential benefits of circumcisions include lower risk of cancer of the penis, lower risk of bladder and kidney infections, and lower risk of transmitting sexual diseases.

The Gomco technique is one of the leading techniques used for circumcisions of newborn infants in the United States. The Gomco technique is well known for its safety record in preventing injury and bleeding.

The Gomco technique is fairly straightforward with proper training. However, clinicians frequently find one step in the Gomco procedure technically difficult. Once the foreskin is over the Gomco bell, it is somewhat technically difficult to pull the foreskin and the handle of the bell through the circular hole of the base plate with only the use of hemostats. Additionally, the use of a safety pin to perform such task poses a risk to both the clinician and newborn infant. Lastly, if too much time is taken manipulating the foreskin and handle of the bell through the hole of the base plate, the foreskin can develop significant swelling.

A need therefore exists for facilitating a critical step of the Gomco technique to ensure safe success.

### BRIEF DESCRIPTION OF THE DRAWINGS

The features of the system, which are believed to be novel, are set forth with particularity in the appended claims. The embodiments herein, can be understood by reference to the following description, taken in conjunction with the accompanying drawings, in the several figures of which like reference numerals identify like elements, and in which:

FIG. 1A depicts an exemplary illustration of a circumcision (circ) clip in accordance with one embodiment;

FIG. 1B illustrates a top view of the circ clip in accordance with one embodiment;

FIG. 2A depicts a front view of the circ clip in accordance with one embodiment;

FIG. 2B depicts a side view of the circ clip in accordance with one embodiment where the clasp tongs are aligned;

FIG. 2B depicts a side view of the circ clip in accordance with one embodiment where the clasp tongs are offset;

FIG. 3A depicts an exemplary illustration of the circ clip prior to use with a Gomco bell in accordance with one embodiment;

FIG. 3B depicts an exemplary illustration of the circ clip used in conjunction with a Gomco bell in accordance with one embodiment;

FIG. 4 shows a closer view of the circ clip used in conjunction with a Gomco bell shown in FIG. 3B; and

FIG. 5 depicts an exemplary illustration of the circ clip used for insertion through a base plate of the Gomco bell in accordance with one embodiment;

### DETAILED DESCRIPTION

Embodiments in accordance with the present disclosure provide a circumcision clip (hereinafter CIRC CLIP) to facilitate a critical step of the circumcision technique to ensure safe success.

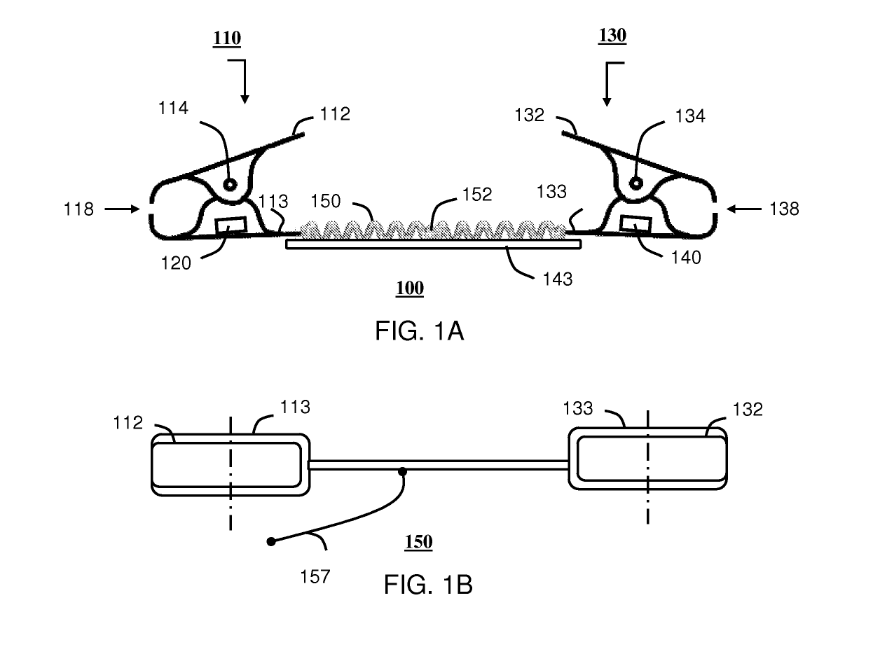

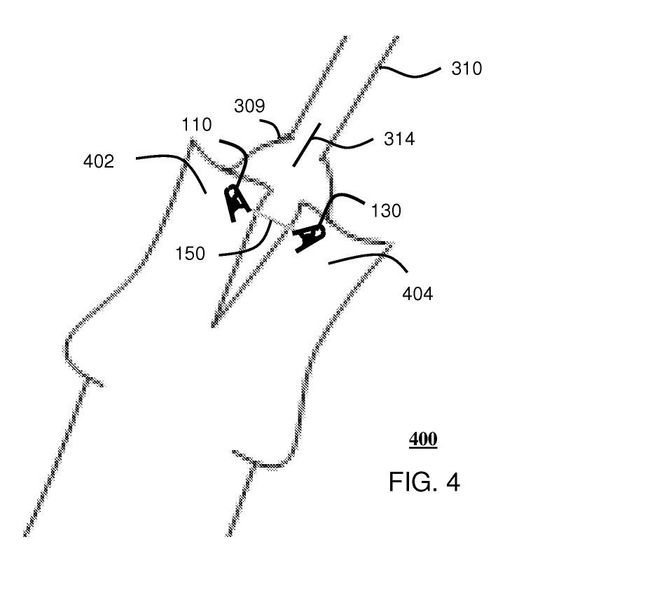

Referring to FIG. 1, an exemplary illustration of the CIRC CLIP 100 is shown according to one embodiment. As illustrated the CIRC CLIP 100 comprises a first clip 110, a second clip 130 and a retracting mechanism 150 that couples the first clip 110 to the second clip 130. The first clip 110 by way of the clamping portion 118 attaches to a first foreskin edge 402 of an incised foreskin 406 during a circumcision procedure herein disclosed as illustrated in FIG. 4 and discussed ahead in further detail. The second clip 130 by way of the clamping portion 138 attaches to a second foreskin edge 404 of the incised foreskin 406. The retracting mechanism 150 respectively permits the first clip 110 and second clip 130 to stretch away and grasp the first foreskin edge 402 and second foreskin edge 404 around the bell 309.

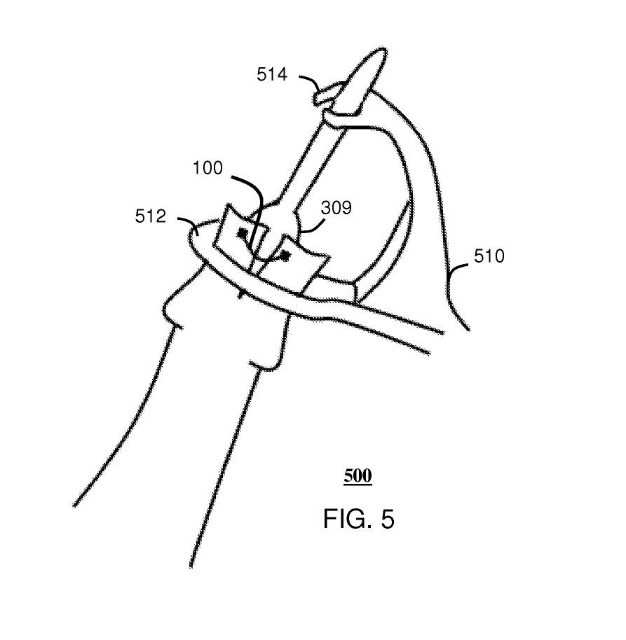

The retracting mechanism 150 in one embodiment is a spring that pulls the first clip 110 and second clip 130 together to a natural separation that approximates a closing of the first 402 and second 404 foreskin edges and securely fastens the first and second foreskin edges over the base of the bell 309. Briefly referring to FIG. 5 and as will be explained ahead in further detail, this retracting mechanism 150 pulls together the first clip 110 the second clip 130 prior to, and during, insertion through a circular opening of a base plate 512 positioned over the bell 309 as part of the circumcision procedure herein disclosed. The first clip 110, second clip 130, and retracting mechanism 150 pull snugly and lay against the bell 309 once the first clip 110 and second clip 130 are fastened to the foreskin edges 402/404. This permits the foreskin along with the CIRC CLIP 100 to then be pulled through the circular hole of the base plate 512.

Returning back to FIG. 1, according to one embodiment, the first clip 110 comprises a spring loaded mechanism 114 with a top lever 112 and a bottom lever 113. The second clip 130 similarly comprises a spring loaded mechanism 134 with a top lever 132 and a bottom lever 133. In lieu of a spring, a flexible plastic housing or other memory retention clip can be used. The retracting mechanism 150 in a first embodiment can be a coiled spring that is tightly wound with a small radius to prevent pinching of the foreskin between coils. In another arrangement, the coiled spring can be loosely wound with a larger radius to minimize pinching of the foreskin between coils. In another embodiment, the retracting mechanism 150 is (or includes) an elastic stretch band 143 comprising rubber along its flat interior length to firmly grip the foreskin and keep the foreskin edges 402 and 404 taut around the bell 309. The retracting mechanism 150 can further comprises a flat underside material 143 to mitigate pinching of the foreskin between coils. The flat under surface is designed to be contiguous with the retracting mechanism 150 to lay flat and contoured against the bell 309 once the first clip and second clip are securely fastened to the foreskin edges. Each clip can further exhibit a slight concavity 161/162 to rest flush around the round contour of the bell 309. The CIRC CLIP 100 can further comprise a first magnet 120 in the first clip and a second magnet 140 in the second clip to magnetically couple to the bell 309. This is to keep the foreskin at a predetermined level 314 (corresponding magnetic strip) on the bell 309 prior to positioning through the hole of the base plate 512 of the Gomco device 510 as illustrated in FIG. 5 and as will be discussed ahead in further detail.

As illustrated in FIG. 1B, the top levers 112/113 in accordance with one embodiment are shorter in length than the bottom levers 132/133 to broadly distribute a downward applied force across the bell and reduce pressure on the foreskin disposed there between on release of the first clip 110 or second clip 130. In accordance with another embodiment, the bottom levers 113/133 are wider in dimension than the top levers 112/132 to further broadly distribute a downward applied force across the Gomco bell and reduce pressure on the foreskin upon release of the first clip or second clip. A line attachment (also called a guider) 157 can be optionally coupled to the retracting mechanism 150 to assist in pulling the CIRC CLIP 100 with attachment to the first 402 and second 404 foreskin through the hole of the base plate 512 of the Gomco device 510 as illustrated in FIG. 5. The line attachment 157 can be a string, a flexible plastic or other construct that is sufficiently flexible to bend and thread through the base plate 512 during the surgical procedure. It may include a detachable mechanism such as a lock and hook to temporarily connect to the retracting mechanism at a predetermined location 152.

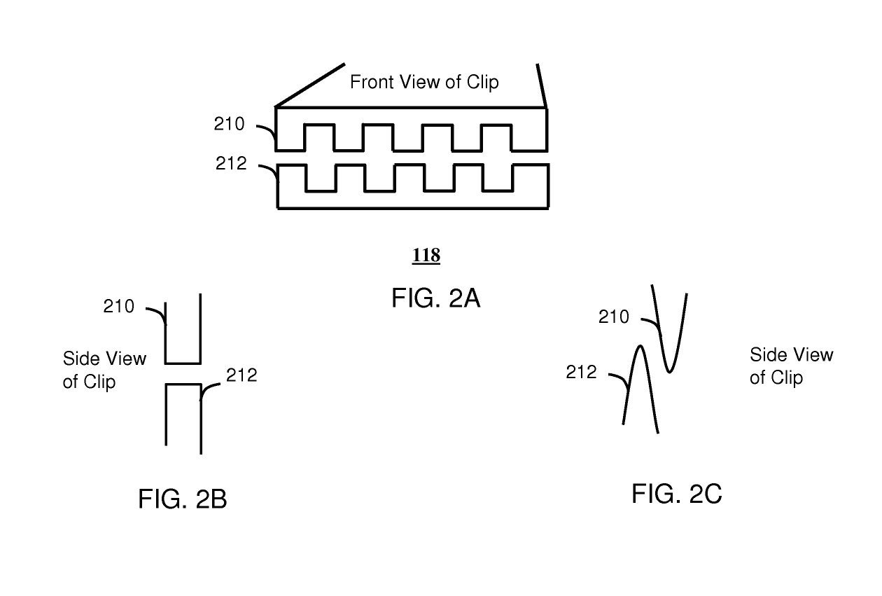

Referring to FIG. 2A, a front view of the clamping portion (118/138) of the first clip 110 and second clip 130 is shown according to one embodiment. The clamping portion can include a top row of teeth 210 and a bottom row of teeth 212. The teeth provide one means for gripping. Other means are herein contemplated, such as rubber teeth or a flat rubber liner for gripping and securing of the foreskin. FIG. 2B shows one arrangement where the teeth are aligned atop one another to provide downward direct force contact. FIG. 2C shows another arrangement where the teeth are rounded and off-centered to apply a shearing force between the teeth to provide grip.

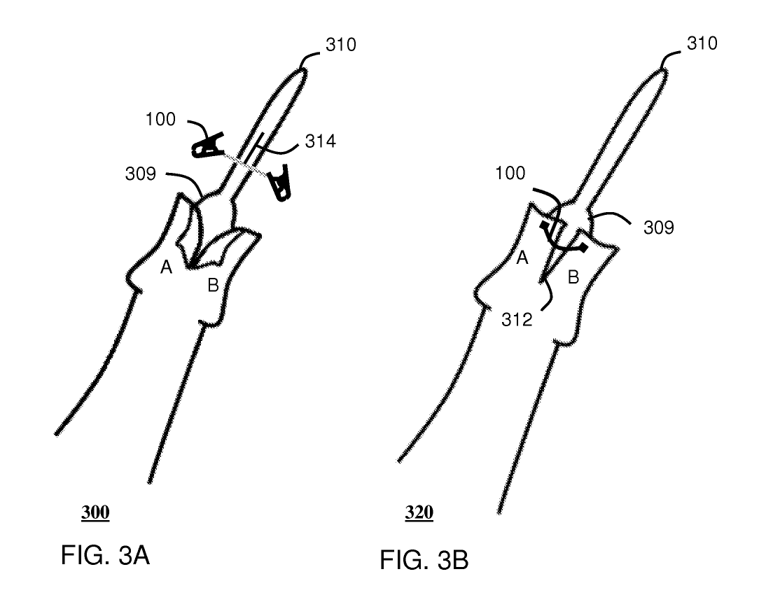

A method that employs the CIRC CLIP 100 is further herein disclosed according to one embodiment. The method is not limited to the steps shown or the order of the steps shown, and may include more or less than the number of steps shown. A workflow of the method is presented below and in accordance with the features of the figures which are believed to be novel, and set forth with particularity in the appended claims. Reference will be made to FIGS. 3-5 for identifying the features and components associated with the method steps.

The method 600 can begin at step 601, at which time a dorsal penile nerve block is administered (optional). At step 602, the patient is prepped and draped in a sterile fashion. At step 603, the foreskin is grasped on either side of the center at approximately 2 o'clock and 10 o'clock by way of a first and second hemostat. A hemostat is a surgical tool that stops bleeding by clamping the blood vessel. At step 604, a third hemostat is used to separate the adhesions between the most inner mucosal layer of the foreskin and the glans of the penis down to the level of the corona. This is done by inserting the hemostat into the preputial ring pointing the tip of the hemostat and tenting the foreskin away (See FIG. 3A) from the glans taking care to avoid the urethral meatus. At step 605, a straight hemostat is then used to create a crush injury on the dorsal aspect of the foreskin (at the midline) to approximately 1 to 1.5 cm from the coronal sulcus. At step 606, the linear crushed skin is cut with scissors resulting in open dorsal edges (see FIG. 3A). Using the two hemostats still in place on the foreskin, the incised foreskin is retracted back (proximally) exposing the glans at step 607. Any additional adhesions are removed by a blunt probe or gauze.

Referring to FIG. 3A, at step 608, the foreskin is pulled back over the glans, and the bell 309 of the Gomco 310 is inserted over the glans and positioned under the foreskin. The foreskin now is unsecured with the dorsal slit 312 and two edges of the foreskin (A and B). The CIRC CLIP (FIG. 1) having the two small clips (e.g., first clip 110 and second clip 130) on either end. The two small clips are connected by the retracting mechanism 150 (e.g. spring) that allows the CIRC CLIP to stretch in order to grasp edge A and edge B of the dorsal slit around the base of the bell. In lieu of a safety pin or large cumbersome hemostats commonly used today, the CIRC CLIP 100 fastens the edges of the dorsal slits 402/404 and pulls them taut over the bell 309.

Once both edges are securely fastened by each side of the clip, the CIRC CLIP 100 is released by the clinician allowing the spring to recoil back to the apparatus' original position as shown in FIG. 3B and thus retracting the dorsal edges A and B together and securely fastening them over the base of the bell 309. The CIRC CLIP 100 lays snugly against the bell 309 once the CIRC CLIP 100 is fastened to the foreskin edges. This allows the clinician to more easily pull the foreskin through the hole of the GOMCO base plate 512.

As previously mentioned, the line attachment 157 (see FIG. 1B) provides a means for threading the CIRC CLIP 100 through the hole. At step 609, the CIRC CLIP 100 attached to the foreskin is then pulled through the circular hole of the Gomco base plate 512 (see FIG. 5.) Once this has been done, the entire handle 310 and base of the bell 309 with the foreskin securely fastened by the CIRC CLIP 100 over the base of the bell 309 will more easily be pulled into and against the circular hole of the Gomco base 512. The CIRC CLIP 100 allows the bell to remain under the foreskin while the clinician quickly pulls the foreskin through the circular hole of the Gomco plate 512. This allows the clinician to avoid risks commonly associated with a safety pin or manual pushing of the foreskin or the cumbersome use of hemostats through the base of the Gomco plate 512.

At step 610, the arms 514 and handle 310 of the bell, the foreskin, and the CIRC CLIP 100 are firmly pulled through the circular hole of the Gomco plate. At step 611, the top plate is attached under the arms of the bell and into the notch of the base plate. A nut is placed onto the base plate screw and tightened. This creates a circular crush injury on the foreskin between the bell 309 and the base plate 512. After waiting approximately 5 minutes the foreskin is excised right above the base plate 512 at step 612. The nut is then removed, followed by the removal of the top 510 and base plate 512. The bell 309 is carefully removed carefully removing the shaft skin (severed foreskin) off of the bell 309.

Upon reviewing the aforementioned embodiments, it would be evident to an artisan with ordinary skill in the art that said embodiments can be modified, reduced, or enhanced without departing from the scope and spirit of the claims described below. Accordingly, the reader is directed to the claims section for a fuller understanding of the breadth and scope of the present disclosure.

While the invention has been described in conjunction with specific embodiments, it is evident that many alternatives, modifications, permutations and variations will become apparent to those of ordinary skill in the art in light of the foregoing description. Accordingly, it is intended that the present invention embrace all such alternatives, modifications, permutations and variations as fall within the scope of the appended claims. While the preferred embodiments of the invention have been illustrated and described, it will be clear that the embodiments of the invention are not so limited. Numerous modifications, changes, variations, substitutions and equivalents will occur to those skilled in the art without departing from the spirit and scope of the present embodiments of the invention as defined by the appended claims.

What is claimed is:

1. A circumcision clip comprising:
a first clip;
a second clip; and
a retracting mechanism that couples the first clip to the second clip, wherein
the first clip attaches to a first foreskin edge of an incised foreskin,
the second clip attaches to a second foreskin edge of the incised foreskin, and
the retracting mechanism respectively permits the first clip and second clip to stretch away and grasp the first foreskin edge and second foreskin edge around a bell, and thereafter pull together the first clip the second clip prior to, and during, insertion through a circular opening of a base plate positioned over the bell.

2. The circumcision clip of claim 1, wherein the retracting mechanism is a spring that pulls the first clip and second clip together to a natural separation that approximates a closing of the first and second foreskin edges and securely fastens the first and second foreskin edges over a base of the bell.

3. The circumcision clip of claim 2, wherein the coiled spring is tightly wound with a small radius to prevent pinching of the foreskin between coils, and the retracting mechanism comprises a flat underside material to mitigate pinching of the foreskin between coils.

4. The circumcision clip of claim 2, wherein the coiled spring is loosely wound with a large radius to minimize pinching of the foreskin between coils.

5. The circumcision clip of claim 2, wherein the retracting mechanism further comprises a flat underside material to mitigate pinching of the foreskin between coils.

6. The circumcision clip of claim 1, wherein the first clip, second clip, and retracting mechanism pull snugly and lay against the bell once the first clip and second clip are fastened to the foreskin edges.

7. The circumcision clip of claim 6, further comprising a first magnet in the first clip and a second magnet in the second clip to magnetically couple to the bell and keep the foreskin at a predetermined level on the bell.

8. The circumcision clip of claim 6, wherein the first clip and the second clip each have a contoured under surface so as to be contiguous with the retracting mechanism to lay flush against the bell once the first clip and second clip are securely fastened to the foreskin edges.

9. The circumcision clip of claim 8, wherein the retracting mechanism is a flat elastic stretch band comprising rubber along its flat interior length to firmly grip the foreskin and keep the foreskin edges taut around the bell.

10. The circumcision clip of claim 8, wherein each of the first clip and second clip is a spring loaded mechanism with a top lever and a bottom lever, where the top lever is shorter in length than the bottom lever to broadly distribute a downward applied force across the bell and reduce pressure on the foreskin disposed there between on release of the first clip or second clip.

11. The circumcision clip of claim 8, wherein the bottom lever is wider in dimension that the top lever to further broadly distribute a downward applied force across the Gomco bell and reduce pressure on the foreskin upon release of the first clip or second clip.

12. A circumcision clip comprising:
a first clip;
a second clip; and
a spring that couples the first clip to the second clip, wherein
the first clip attaches to a first foreskin edge of an incised foreskin,
the second clip attaches to a second foreskin edge of the incised foreskin, and
the spring respectively permits the first clip and second clip to stretch away and grasp the first foreskin edge and second foreskin edge around a bell, and thereafter pull together the first clip the second clip prior to, and during, insertion through a circular opening of a base plate positioned over the bell.

13. The circumcision clip of claim 12, where each of the first clip and the second clip comprise a flat contoured underside bottom lever that rests against a convex curvature of the bell.

14. The circumcision clip of claim 12, where the spring comprises a flat contoured underside to mitigate pinching of the foreskin between coils.

15. The circumcision clip of claim 12, where the first clip is soldered to a first end of the spring while the second clip is soldered to a second end of the spring.

16. The circumcision clip of claim 12, wherein the first clip and the second clip are spring loaded.

17. A circumcision clip comprising:
a first clip;
a second clip; and
a spring that couples the first clip to the second clip, wherein
the first clip attaches to a first foreskin edge of an incised foreskin,
the second clip attaches to a second foreskin edge of the incised foreskin,
the spring respectively permits the first clip and second clip to stretch away and grasp the first foreskin edge and second foreskin edge around a bell, and thereafter pull together the first clip the second clip prior to, and during, insertion through a circular opening of a base plate positioned over the bell; and
a guider coupled to the retracting mechanism to assist in pulling the first clip and the second clip through a hole of the base plate.

18. The circumcision clip of claim 17, wherein the retracting mechanism is a spring that pulls the first clip and second clip together to a natural separation that approximates a closing of the first and second foreskin edges and securely fastens the first and second foreskin edges over a base of the bell.

19. The circumcision clip of claim 17, wherein the first clip and the second clip are spring loaded.
